(19) United States
(12) Patent Application Publication
(10) Pub. No.: US 2025/0049680 A1
(43) Pub. Date: Feb. 13, 2025

(54) COMPOSITIONS AND METHODS FOR TREATING DENTIN CARIES

(71) Applicant: Rutgers, The State University of New Jersey, New Brunswick, NJ (US)

(72) Inventor: Mohammad Ali SAGHIRI, Lyndhurst, NJ (US)

(21) Appl. No.: 18/800,641

(22) Filed: Aug. 12, 2024

Related U.S. Application Data
(60) Provisional application No. 63/531,963, filed on Aug. 10, 2023.

(51) Int. Cl. A61K 8/55; A61K 8/19; A61K 8/25; A61K 8/27; A61Q 11/00

(52) U.S. Cl. CPC A61K 8/55; A61K 8/19; A61K 8/25; A61K 8/27; A61Q 11/00

(57) ABSTRACT

The present disclosure provides for oral compositions and method of using the same for treating dentin using an orally acceptable salt which includes a divalent metal cation and an anionic counterion, such as an ascorbic acid phosphate counterion and a silane coupling agent. The methods provide for remineralization and reinforcing of dentin by doping with the divalent cation, thereby preventing and/or reducing the occurrence of dental caries

### CROSS-REFERENCE TO RELATED APPLICATIONS

This application claims the benefit of U.S. Provisional Application No. 63/531,963, filed on 10 Aug. 2023, which application is incorporated herein by reference.

### STATEMENT AS TO FEDERALLY SPONSORED RESEARCH

This invention was made with government support under Grant No. 2312680 awarded by the National Science Foundation. The government has certain rights in the invention.

### BACKGROUND

Dentin, a hard and slightly compressible oral tissue, contains approximately 70% inorganic elements, 20% organic elements, and 10% water. It is characterized by long, thin tubules surrounded by inorganic peritubular dentin with a mineral content of over 90%.

Dental caries is a prevalent pathological change of dentin driven by disruption of a demineralization-remineralization balance by acidogenic bacteria. Root dentin is particularly susceptible to caries development, with a rate of mineral loss twice as fast as in enamel. Carious dentin has a reduced crystallinity and lower mineral content, with changes in magnesium levels indicating early demineralization and loss of a peritubular dentin matrix. Clinically, carious dentin is softer than healthy dentin and exhibits lower tensile strength due to mineral loss in intertubular dentin. Additionally, carious dentin shows histopathological changes that may lower bonding efficacy.

Inorganic trace mineral levels affect dentin structure and mechanical properties, and ionic doping may drive such changes in dentin's hydroxyapatite crystalline matrix [Ca₁₀(PO₄)₆(OH)₂]. Previous studies on mechanical properties of hydroxyapatite as a result of ionic doping have been done on synthetic hydroxyapatite instead of natural biological materials, and studies of natural materials mainly focused on bone and enamel. In comparison, studies on dentin have been very limited. Despite studies on the effects of trace elements in enamel, leading to the development of innovative mouth rinses and washes rich in certain ions that improve enamel strength, there remains a significant lack of research on the impact of inorganic trace minerals (IoTM) on dentin. Dentin is covered by enamel, making it challenging for elements in mouth rinses to penetrate all the way through to the dentin. Recent studies have started investigating the effects of trace elements in dentin, but there is insufficient data on the repercussions of IoTM on dentinal hydroxyapatite (HAp) and its mineralization and mechanical properties. Particularly intriguing is the apparent disconnect between the structure and mechanical properties of dentin. Given the differences in the shape and composition of HAp crystals in dentin compared to bone and enamel, it is reasonable to expect that dentin would behave differently when exposed to IoTM. Furthermore, dentin's heterogeneous and porous structure may make it more susceptible to undergoing cationic substitutions.

There thus remains a need to treat dentin caries and to alleviate some of the above problems, to at least some extent.

### SUMMARY

In some embodiments, the present disclosure relates to oral compositions including: (i) an effective amount of an orally acceptable salt including an orally acceptable divalent metal cation and an anionic counterion, wherein the anionic counterion includes an ascorbic acid phosphate counterion; and (ii) a silane coupling agent. In some embodiments, the divalent metal cation is selected from strontium (Sr), barium (Ba), zinc (Zn), magnesium (Mg), manganese (Mn), or any combination thereof. In some embodiments, the Mg is present in a concentration of 0.18 mM. In some embodiments, the Zn is present in a concentration of 5.3 μM. In some embodiments, the Mn is present in a concentration of 2.2×10-8 M. In some embodiments, the Sr is present in a concentration of 1.8 μM. In some embodiments, the Ba is present in a concentration of 1.9 μM. In some embodiments, the divalent metal cation comprises from 10% to 15% by total weight of the composition. In some embodiments, the ascorbic acid phosphate counterion comprises L-ascorbic acid-2-phosphate (AA2P), L-ascorbic acid-3-phosphate (AA2P), or a combination thereof. In some embodiments, the ascorbic acid phosphate counterion is AA2P. In some embodiments, the AA2P comprises from 5% to 10% by total weight of the composition. In some embodiments, the silane coupling agent comprises 3-methacryloxypropyltrimethoxysilane (MPTS). In some embodiments, the MPTS comprises from 0.5% to 2% by total weight of the composition. In some embodiments, the composition, further comprises a nano-zeolite. In some embodiments, the nano-zeolite comprises from 1% to 5% by total weight of the composition. In some embodiments, the composition is formulated as a toothpaste, a gel, a dental cream, a mouth-wash, a mouth-rinse, a chewing gum, a lozenge, or a food product.

Further, in some embodiments, provided herein are methods for treating a dentin, the method including: contacting the dentin with an oral composition including (i) an effective amount of an orally acceptable salt including an orally acceptable divalent metal cation and an anionic counterion, wherein the anionic counterion includes an ascorbic acid phosphate counterion; and (ii) a silane coupling agent; allowing the oral composition to remain in contact with the dentin for a predetermined duration; and rinsing the dentin, wherein the treatment results in at least one of the following: remineralization of the dentin, improvement of surface microhardness, increase in diametral tensile strength (DTS), increase in compressive strength of the dentin compared to untreated dentin or any combination thereof. In some embodiments, the contacting lasts from 1 minute to 5 minutes, and is followed by rinsing the dentin with distilled water. In some embodiments, the treating comprises remineralization the dentin by doping with the divalent cation. In some embodiments, the treating comprises penetrating and reinforcing the dentin against acid dissolution compared to an untreated dentin. In some embodiments, the treating comprises improving a surface microhardness of the dentin compared to an untreated dentin. In some embodiments, the surface microhardness is improved at a depth of from 100 μm to 500 μm. In some embodiments, the treating comprises increasing a diametral tensile strength (DTS) of the dentin compared to an untreated dentin In some embodiments, the treating comprises increasing a compressive strength of the dentin compared to an untreated dentin

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a flowchart of an experimental design of some embodiments of the disclosure.

FIG. 2 is a flowchart detailing the steps taken to make cationic exchange solutions of embodiments of the disclosure.

FIG. 3A-FIG. 3F show: (i) Box plots of means and standard deviations showing weight loss (%) for six groups of the disclosure (FIG. 3A); (ii) box plots of means and standard deviations showing DTS (Mpa) for the six groups (FIG. 3B); (iii) box plots of means and standard deviations showing compressive strength (Mpa) for the six groups (FIG. 3C); (iv) box plots of means and standard deviations showing surface microhardness at 100 and 500 μm depths for the six groups (FIG. 3D); (v) mean and 95% confidence interval of microhardness for the groups by depth (FIG. 3E); and (vi) pixel intensity of dentin specimens at 500 μm depth, where the different superscript letters in each column represent significant differences (FIG. 3F).

FIG. 4A-FIG. 4E show EDS dot maps and histogram analyses of dentin specimens at 1000× for (i) Mg (FIG. 4A); (ii) Zn (FIG. 4B); (iii) Mn (FIG. 4C); (iv) Sr (FIG. 4D); and (v) Ba (FIG. 4E).

### DETAILED DESCRIPTION

Without wishing to be bound by theory, Applicant surprisingly found that dentin strength and integrity could be enhanced using inorganic rich trace metal solutions as described herein to aid in preventing tooth fractures and structural issues, leading to improved dental health. Additionally, the compositions described herein could enhance natural defense mechanisms against decay-causing bacteria, reducing the risk of cavities and invasive dental treatments, while preserving the vitality of dental pulp. Applicant's studies have shown promising results, indicating that IOTM-rich solutions can fortify dentin samples, to improve dentinal quality. These findings provide novel treatments for dental diseases and disorders.

Applicants of the present disclosure evaluated in vitro remineralization effects of five fortifying solutions (0.1 mol/L AA2P) salts of strontium (Sr), barium (Ba), zinc (Zn), magnesium (Mg), and manganese (Mn), containing a large non-exchangeable anionic counterion of AA2P on human premolar dentin caries. Applicants surprisingly found that the trace elements Sr, Ba, Zn, Mg, and Mn can significantly impact dentinal hydroxyapatite quality and reduce the occurrence of caries. Mg and Zn were found to improve dentin mineralization and Sr possessed antimicrobial properties, showing great potential in anti-caries treatment along with Ba. The role of Mn was dependent on the amount used and may propagate or reduce caries. Applicant surprisingly found useful compositions and methods to control the physical properties of carious dentin.

In some embodiments, the oral composition comprises (i) an effective amount of an orally acceptable salt. In some embodiments, the orally acceptable salt comprises an orally acceptable divalent metal cation. In some embodiments, the divalent metal cation is selected from strontium (Sr), barium (Ba), zinc (Zn), magnesium (Mg), manganese (Mn), or any combination thereof. In some embodiments, the divalent metal cation is strontium (Sr). In some embodiments, the divalent metal cation is barium (Ba). In some embodiments, the divalent metal cation is zinc (Zn). In some embodiments, the divalent metal cation is magnesium (Mg). In some embodiments, the divalent metal cation is manganese (Mn). In some embodiments, the divalent metal cation comprises Sr and Ba. In some embodiments, the divalent metal cation comprises Sr and Zn. In some embodiments, the divalent metal cation comprises Sr and Mg. In some embodiments, the divalent metal cation comprises Sr and Mn. In some embodiments, the divalent metal cation comprises Ba and Zn. In some embodiments, the divalent metal cation comprises Ba and Mg. In some embodiments, the divalent metal cation comprises Ba and Mn. In some embodiments, the divalent metal cation comprises Zn and Mg. In some embodiments, the divalent metal cation comprises Zn and Mn. In some embodiments, the divalent metal cation comprises Mg and Mn. In some embodiments, the divalent metal cation comprises Sr, Ba, and Zn. In some embodiments, the divalent metal cation comprises Sr, Ba, and Mg. In some embodiments, the divalent metal cation comprises Sr, Ba, and Mn. In some embodiments, the divalent metal cation comprises Sr, Zn, and Mg. In some embodiments, the divalent metal cation comprises Sr, Zn, and Mn. In some embodiments, the divalent metal cation comprises Sr, Mg, and Mn. In some embodiments, the divalent metal cation comprises Ba, Zn, and Mg. In some embodiments, the divalent metal cation comprises Ba, Zn, and Mn. In some embodiments, the divalent metal cation comprises Ba, Mg, and Mn. In some embodiments, the divalent metal cation comprises Zn, Mg, and Mn. In some embodiments, the divalent metal cation comprises Sr, Ba, Zn, and Mg. In some embodiments, the divalent metal cation comprises Sr, Ba, Zn, and Mn. In some embodiments, the divalent metal cation comprises Sr, Ba, Mg, and Mn. In some embodiments, the divalent metal cation comprises Sr, Zn, Mg, and Mn. In some embodiments, the divalent metal cation comprises, Ba, Zn, Mg, and Mn. In some embodiments, the divalent metal cation comprises Sr, Ba, Zn, Mg, and Mn.

In some embodiments, the divalent metal cation comprises from 10% to 15% by total weight of the composition. In some embodiments, the divalent metal cation comprises 10 weight % of the composition. In some embodiments, the divalent metal cation comprises 11 weight % of the composition. In some embodiments, the divalent metal cation comprises 12 weight % of the composition. In some embodiments, the divalent metal cation comprises 13 weight % of the composition. In some embodiments, the divalent metal cation comprises 14 weight % of the composition. In some embodiments, the divalent metal cation comprises 15 weight % of the composition.

In some embodiments, the Mg is present in a concentration of 0.18 mM. In some embodiments, the Zn is present in a concentration of 5.3 μM. In some embodiments, the Mn is present in a concentration of 2.2×10-8 M. In some embodiments, the Sr is present in a concentration of 1.8 μM. In some embodiments, the Ba is present in a concentration of 1.9 μM.

In some embodiments, the oral composition comprises an anionic counterion. In some embodiments, the anionic counterion comprises an ascorbic acid phosphate counterion. In some embodiments, the ascorbic acid phosphate counterion comprises L-ascorbic acid-2-phosphate (AA2P), L-ascorbic acid-3-phosphate (AA2P), or a combination thereof. In some embodiments, the ascorbic acid phosphate counterion comprises L-ascorbic acid-2-phosphate (AA2P) or L-ascorbic acid-3-phosphate (AA2P). In some embodiments, the ascorbic acid phosphate counterion comprises L-ascorbic acid-2-phosphate (AA2P) and L-ascorbic acid-3-phosphate (AA2P). In some embodiments, the ascorbic acid phosphate counterion comprises L-ascorbic acid-2-phosphate (AA2P). In some embodiments, the ascorbic acid phosphate counterion comprises L-ascorbic acid-3-phosphate (AA2P).

In some embodiments, the ascorbic acid phosphate counterion is AA2P. In some embodiments, the AA2P comprises from 5% to 10% by total weight of the composition. In some embodiments, the AA2P comprises 5 weight % of the composition. In some embodiments, the AA2P comprises 6 weight % of the composition. In some embodiments, the AA2P comprises 7 weight % of the composition. In some embodiments, the AA2P comprises 8 weight % of the composition. In some embodiments, the AA2P comprises 9 weight % of the composition. In some embodiments, the AA2P comprises 10 weight % of the composition.

In some embodiments, the oral composition comprises a silane coupling agent. In some embodiments, the silane coupling agent comprises 3-methacryloxypropyltrimethoxysilane (MPTS). In some embodiments, the MPTS comprises from 0.5% to 2% by total weight of the composition. In some embodiments, the MPTS comprises 0.5 weight % of the composition. In some embodiments, the MPTS comprises 1 weight % of the composition. In some embodiments, the MPTS comprises 1.5 weight % of the composition. In some embodiments, the MPTS comprises 2 weight % of the composition.

In some embodiments, the composition, further comprises a nano-zeolite. In some embodiments, the nano-zeolite comprises from 1% to 5% by total weight of the composition. In some embodiments, the nano-zeolite comprises 1 weight % of the composition. In some embodiments, the nano-zeolite comprises 2 weight % of the composition. In some embodiments, the nano-zeolite comprises 3 weight % of the composition. In some embodiments, the nano-zeolite comprises 4 weight % of the composition. In some embodiments, the nano-zeolite comprises 5 weight % of the composition.

In some embodiments, the composition is formulated as a toothpaste. In some embodiments, the composition is formulated as a gel. In some embodiments, the composition is formulated as a dental cream. In some embodiments, the composition is formulated as a mouth-wash. In some embodiments, the composition is formulated as a mouth-rinse. In some embodiments, the composition is formulated as a chewing gum. In some embodiments, the composition is formulated as a lozenge. In some embodiments, the composition is formulated as a food product.

In some embodiments, of the present disclosure the molecular weights of the individual components in the formulation are as follows: (i) approximately 258.11 g/mol L-ascorbic acid 2-phosphate (AA2P); and (ii) 24.305 g/mol magnesium ion (Mg²⁺), 65.38 g/mol zinc ion (Zn²⁺), 54.938 g/mol manganese ion (Mn²⁺), 87.62 g/mol strontium ion (Sr²⁺), and/or 137.327 g/mol barium ion (Ba²⁺). When these ions form complexes with AA2P, the molecular weights of the resulting compounds increase. In some embodiments, the overall molecular weight of the final composition of within the 200-1000 g/mol range, and is achieved by balancing the ratios of each component in the formulation. In some embodiments, effective penetration into dentinal tubules can be obtained by using formulations comprising AA2P-metal cation complexes as follows: (i) AA2P-Mg Complex: 282.415 g/mol; (ii) AA2P-Zn Complex: 322.49 g/mol; (iii) AA2P-Mn Complex: 312.048 g/mol; (iv) AA2P-Sr Complex: 342.73 g/mol; and/or (v) AA2P-Ba Complex: 392.437 g/mol.

In some embodiments, the method for using the disclosed composition comprises a surgical procedure, such as a root canal treatment.

In some embodiments, the method for treating a dentin comprises contacting the dentin with an oral composition described herein. In some embodiments, the contacting lasts from 1 minute to 5 minutes. In some embodiments, the contacting lasts for 1 minute. In some embodiments, the contacting lasts for 2 minutes. In some embodiments, the contacting lasts for 3 minutes. In some embodiments, the contacting lasts for 4 minutes. In some embodiments, the contacting lasts for 5 minutes. In some embodiments, the contacting is followed by rinsing the dentin. In some embodiments, the rinsing is with distilled water. In some embodiments, the treating comprises remineralization the dentin by doping with the divalent cation. In some embodiments, the treating comprises penetrating and reinforcing the dentin against acid dissolution compared to an untreated dentin. In some embodiments, the treating comprises improving a surface microhardness of the dentin compared to an untreated dentin. In some embodiments, the surface microhardness is improved at a depth of from 100 μm to 500 μm. In some embodiments, the treating comprises increasing a diametral tensile strength (DTS) of the dentin compared to an untreated dentin In some embodiments, the treating comprises increasing a compressive strength of the dentin compared to an untreated dentin

In some embodiments, the methods comprises several steps to ensure effective penetration of the composition into the dentinal tubules and the enhancement of the mechanical properties of the dentin. In some embodiments, the method comprises: (i) preparing a root canal in a standard manner known in the art, ensuring that all infected or damaged tissue is removed; (ii) removing a smear layer, which forms on the surface of dentin during mechanical preparation to allow the composition described herein to effectively penetrate the dentin; for example, by using an EDTA solution or a similar agent to dissolve the smear layer; (iii) flushing the root canal with distilled water to remove any residual debris and the EDTA solution, wherein distilled water is used to prevent any unwanted chemical interactions that could affect the composition's efficacy; (iv) applying the disclosed composition containing the AA2P salts of Mg, Zn, Mn, Sr, and/or Ba to the dentin surface; and (v) allowing the composition to sit in the root canal for a duration of 1 to 5 minutes.

In some embodiments, the time frame ensures that the composition penetrates the dentinal tubules and interacts with the hydroxyapatite structure. In some embodiments, the method further comprises flushing the root canal with distilled water after the composition has been allowed to sit for a sufficient time. In some embodiments, this flushing step ensures removal of any excess composition that has not penetrated the dentin, preventing potential adverse effects or interactions with a scaling material. In some embodiments, distilled water is chosen for the flushing step to maintain the purity of the procedure, as it does not introduce any additional ions or contaminants that could interfere with the treatment. In some embodiments, once the root canal has been flushed and dried, it is then sealed. In some embodiments, the choice of sealer is critical to the success of the treatment. In some embodiments, the method provides for a ceramic-based endodontic sealer. In some embodiments, the method provides for a polymer-based endodontic sealer. In some embodiments, ceramic-based sealers offer excellent biocompatibility and mechanical properties, making them ideal for long-term scaling and reinforcement of the treated dentin. In some embodiments, polymer-based sealers provide flexibility and case of use, with the added benefit of being able to bond well with various materials used in dental restorations. In some embodiments, after the scaling procedure, the tooth can be restored using appropriate materials, depending on the clinical situation. In some embodiments, the enhanced mechanical properties of the dentin treated with the compositions provided herein should contribute to the longevity and success of the restoration.

In some embodiments, the method for treating dentin involves: (i) preparing the dentin surface by removing the smear layer using an EDTA solution; (ii) flushing with distilled water to remove debris and residual agents; (iii) applying the AA2P-metal cation complex solution with a molecular weight of 332.024 g/mol; (iv) allowing the solution to penetrate for 1-5 minutes; (v) flushing with distilled water to remove excess composition; and (vi) sealing the treated dentin using a ceramic-based or polymer-based endodontic sealer to ensure longevity and prevent bacterial infiltration.

In some embodiments, the dental composition comprises a stabilizing component of L-ascorbic acid 2-phosphate (AA2P) forming complexes with metal cations selected from Mg²⁺, Zn²⁺, Mn²⁺, Sr²⁺, and Ba²⁺. In some embodiments, the composition's molecular weight is approximately 332.024 g/mol, ensuring effective penetration into dentinal tubules. In some embodiments, the composition includes the metal cations in equal molar ratios, contributing to an overall balance in the molecular weight and viscosity, optimized for flow and penetration. In some embodiments, the composition comprises: 5-10% by weight L-ascorbic acid 2-phosphate (AA2P): 10-15% by weight metal cations selected from Mg²⁺, Zn²⁺, Mn²⁺, Sr²⁺, and/or Ba²⁺; 0.5-2% by weight of a silane coupling agent, such as 3-methacryloxypropyltrimethoxysilane (MPTS); and a balance of distilled water to 100%

In some embodiments, the silane coupling agent, such as MPTS, is included in the compositions described herein to improve the adhesion of the dentin compositions to the dentin surface. In some embodiments, the specified range of 0.5-2% by weight of the silane coupling agent is optimal for enhancing bond strength without negatively affecting the mechanical properties of the compositions or the activity of the compositions.

In some embodiments, the compositions provided herein are applied to the dentin surface following the removal of a smear layer. In some embodiments, the compositions are allowed to penetrate dentinal tubules for 1-5 minutes, ensuring strong adhesion between the treated dentin and subsequent dental materials. In some embodiments, the compositions are then flushed with distilled water before sealing with a ceramic-based or polymer-based sealer.

In some embodiments, a nano zeolite is added to the compositions provided herein to provide controlled ion release, for example of calcium and fluoride, which aids in remineralization of dentin. In some embodiments, a range of 1-5% by weight ensures that the zeolite is present in sufficient quantities to be effective without overwhelming the formulation or causing stability issues. In some embodiments, the combination of MPTS and nano zeolite in the compositions provided herein offers both enhanced adhesion and improved remineralization, providing a comprehensive solution for dental restoration. In some embodiments, the nano zeolite within the composition gradually releases ions, supporting a remineralization process, while the MPTS ensures strong bonding.

In some embodiments, the composition comprises 5-10% by weight L-ascorbic acid 2-phosphate (AA2P); 10-15% by weight metal cations selected from Mg²⁺, Zn²⁺, Mn²⁺, Sr²⁺, and/or Ba²⁺; 0.5-2% by weight of a silane coupling agent such as MPTS; 1-5% by weight of a nano zeolite; and a balance of distilled water to 100%.

In some embodiments, the range for MPTS is from 0.5% to 2% by weight. This range is chosen to ensure that the silane coupling agent effectively enhances adhesion between the dentin compositions and the dentin surface without negatively impacting the composition's viscosity or stability. At concentrations below 0.5%, the adhesion enhancement may be insufficient, while concentrations above 2% may not provide additional benefits and could potentially interfere with the composition's mechanical properties.

In some embodiments, the range for nano zeolite is from 0.5% to 2% by weight. This range ensures that the nano zeolite can effectively contribute to controlled ion release, particularly of calcium and fluoride, which are critical for remineralization. At this concentration, the nano zeolite also provides antimicrobial benefits without overwhelming the composition or negatively affecting its stability. Concentrations below 0.5% may not deliver the desired remineralization effects, while concentrations above 2% could lead to issues with homogeneity or consistency in the composition.

In some embodiments, the inclusion of MPTS as a silane coupling agent improves the adhesion of the dentin vaccine to the dentin surface, enhancing the durability and effectiveness of the treatment. Nano zeolite contributes to controlled ion release, which supports the remineralization of dentin and offers additional antimicrobial protection. Together, these additives enhance the overall functionality of the dentin compositions described herein without compromising its primary purpose of reinforcing and protecting dentin. Both additives are compatible with the composition and do not interfere with the activity of the composition, ensuring that it remains effective in its intended use.

The following are examples of some embodiments of the present invention. It is understood that these examples are merely illustrative and do not limit the scope of the present invention.

### Examples

After establishing that inorganic trace minerals (IoTM) were dysregulated in dentin, Applicant aimed to examine if extrinsic supplementation could enhance dentinal mechanical properties. Specimens were treated with L-ascorbic acid 2-phosphate (AA2P) salts of IoTM as a solute carrier for the elements for 5 minutes. The following findings were observed, as described below.

### Residual Stress

Residual stress differences were significant (p<0.05) between treated and untreated groups at both 0° and 90°. Additionally, Sr-containing compositions generated compressive residual stress whereas Zn-containing compositions generated tensile residual stress.

The specimens underwent various tests after being subjected to pH cycling to mimic caries in dentin, including: (a) Diametral Tensile Strength (DTS) tests, where treated specimens exhibited significantly increased DTS (p<0.05) compared to an untreated control; (b) solubility tests, where dentin specimens were immersed in butyric acid (pH 4.4 for 24 h), and the weight difference before and after immersion was significant (p<0.05) for the treated groups compared to the untreated groups; (c) microhardness tests measured at depths of 100 and 500 μm, revealing significant differences between the treated and control groups at both depths (p<0.05); and (d) compressive strength tests, where treated specimens showed significant differences when compared to the untreated control group (p<0.05). These studies confirmed that IOTM-containing compositions play a crucial role in dentinal mechanical properties and indicates their potential for improving dentin's mechanical properties through extrinsic supplementation, while also cementing AA2P salts as ideal carriers to improve dentinal properties.

Applicants further developed modeling using a predictive ANN model in MATLAB that could integrate all the findings to determine an optimal exposure time for achieving desired dentinal properties. Additionally, Applicant sought to explore potential combinations of specific elements that could lead to favorable outcomes. To achieve this, a preliminary study was conducted using the predictive ANN model, designed to assess the effects of Mg, Zn, and Sr on dentin's mechanical properties based on trace element intervention and the duration of exposure (0, 1, 2, 5, and 10 mins). The ANN model demonstrated high accuracy in predicting test data, yielding a mean percentage relative error of 0.1004% for tubular density, 0.0733% for RFR, and 0.0741% for microhardness. This ANN model serves as a foundation for developing a comprehensive model predicting the mechanical properties of treated dentin.

Without wishing to be bound by theory, Applicant's studies have revealed that dysregulation of inorganic trace minerals (IoTM) results in significant changes in dentinal hydroxyapatite's physical and mechanical properties. This advancement holds great promise in aiding individuals suffering from dental ailments, preventing caries, and addressing oral consequences of systemic diseases using differently sized cations in IoTM-doped compositions (with trace metals such as Mn, Mg, Zn, Sr, and/or Ba). HAp directly affects the residual stress resulting in changes in the dentin crystal structure (crystal symmetry, lattice constants, atomic locations, and occupancy factors), texture, crystallinity, and crystallite size. Dentin can be reached by applying the compositions described herein during root canal irrigation to ensure they effectively reach the dentin.

Dentin specimens were treated with the compositions described herein for different time durations (1, 2, 5, and 8 mins; 8 minutes being a saturation point beyond which substitution does not affect mechanical properties based on preliminary studies), with a saline solution used for a control group (pH ˜6.9-7.4, depending on the cation). The durations were selected based on the average time used for endodontic irrigants.

### IoTM Distribution

Thin sections of the HAp material can be prepared using focused ion beam (FIB) or ultramicrotomy techniques. A high-resolution TEM (HR-TEM) can be used to obtain detailed images of the HAp sample, enabling visualization of its microstructure and morphology at high magnification. Electron energy-loss spectroscopy (EELS) analysis can be performed on the HAp sample within the TEM instrument to determine the elemental composition and chemical bonding. Elemental mapping can be conducted by collecting EELS spectra at different locations across the HAp sample. Elemental maps can provide insights into the distribution of trace elements within the structure. The elemental distribution can be correlated with the observed microstructural features to gain a comprehensive understanding of the relationship between trace elements and the crystal lattice of HAp.

### Substitution Position of IoTM.

X-ray diffraction (XRD) data can be acquired using a powder diffractometer. Specimens are powdered to avoid complications from anisotropic grain growth in the intact samples. Rietveld analysis of the XRD data informs whether the IoTM is substitutional based on changes to the lattice parameters and, if changes did occur, on which cation site the substitution occurred.

### Residual Stress.

Cationic substitution may lead to changes in residual stress. Stress magnitude and distribution by can be measured using XRD (Mn X-ray source, 1 mm aperture) as well as by slitting crack compliance, a destructive mechanical strain release technique, using whole dentin disk samples. In the slitting method, a rectangular sample with a strain gauge applied to the back side will be wired to the electrical discharge machine to cut through a sample to measure strain relief. Finite element analysis can be used to create the stress profile through the depth of a sample. However, the technique's accuracy is limited at the surface. In contrast, XRD is best for surface measurements with very high accuracy. Data can be acquired using a sine-squared psi method to indicate residual stress, modulus, and Poisson ratio. Depth, magnitude, and uniformity of the compressive residual stresses produced by shock peening can also be determined. In-depth XRD stress measurements can be made by creating local surfaces using electro-polishing. Pit depths can be measured using a drop gauge. XRD combined with etching and corrections for stress relaxation vs. depth can provide accurate results to depths of 1 to 2 mm. The residual stress in the surface and sub-surface of dentin apatite (as deep as 12 mm) can be determined.

### Crystal Properties of IoTM-Doped Hap

### Crystallite Size

Whole samples can be used to determine crystal size using wide angle X-ray scattering (WAXS) using the Halder-Wagner model. The analysis will clarify a volume fraction of a disordered phase in the crystals which will serve as a lower bound for the crystal size. WAXS data can be further analyzed using an atomistic model of the dentin apatite nanocrystals using the Debye scattering equation (DSE) to obtain the crystals' coherent domain size. The programs can account for the platy morphology of the crystals, the anisotropic lattice strain caused by substitutions, and the amorphous scattering component. Using calibration parameters and X-ray wavelength, a radial distance (R) can be converted into lattice d-spacing via Bragg's law. Experiments can provide information regarding the size and shape of a crystalline core. The samples can then be powdered to determine the average crystal size using XRD also using the Halder-Wagner model, to account for any anisotropy whole samples may show.

### Morphology

Crystal morphology (isotropic or anisotropic) can be determined using high-resolution transmission electron microscopy (HR-TEM) and WAXS using whole samples. HR-TEM can provide clear localized crystallite pictures, and WAXS can help provide an average of the entire sample. WAXS data can be analyzed to obtain lattice information. WAXS diffraction rings can identify a peak center for all measurable peaks (radial distance) at various azimuthal angles, R(η), using a pseudo-Voigt model. Using calibration parameters and the X-ray wavelength, R can be converted into lattice d-spacing via Bragg's law. D-spacing parameters can be used to determine how the balance of IoTM in dentin apatite affects the internal lattice of the crystals and its potential effects on the lattice's internal stress that can lead to changes in mechanical behavior. Instrumentally corrected FWHM (ΔR) of the (002) peak can be measured using the fit of the diffraction peaks. These values can be converted to Δ2θ using Δ2θ=ΔR/z, where z is the specimen-to-detector distance and recorded as a measure of crystallinity.

### Symmetry

Crystal symmetry can be determined using XRD on powdered samples. Crystal symmetry will indicate if IoTM changes a HAp crystal structure from hexagonal to monoclinic and therefore changes its mechanical properties.

### Growth Direction

Crystal growth direction can be observed on whole samples using HR-TEM along with WAXS. TEM can indicate crystallographic direction and relation to tubules and if the growth changes after IoTM. WAXS can provide an average picture of the sample.

### Crystalline or Amorphous Distribution

The crystalline and amorphous distribution can be found using small-angle x-ray scattering (SAXS) with the powdered sample at a distance of 1.4 m. SAXS data can be analyzed to measure an overall morphology, accounting for crystalline and amorphous phases. Its shape can be determined by assuming a disc-form function and fitting theoretical values with variable thicknesses and diameters to the experimental results using least squares to measure a representative diameter and actual thickness.

Mineral alignment and crystal packing effects may complicate the analysis of the WAXS data. If the preferential alignment of the crystals in dentin becomes a problem, the mounting stage can be modified to rotate during XRD acquisition to minimize alignment effects. Crystal packing can be an issue with the Debye scattering equation (DSE) when a high level of interaction between crystals obscures individual crystal morphologies. If so, the dentin can be ground and crushed before analysis, reducing inter-crystalline interactions. It is possible that the tubule size and density will dominate a SAXS pattern and make it difficult to extract expected morphological information. These effects can be mitigated by grinding samples into a powder.

### Experimental Studies to Elucidate the Sensitivity of HAp Mechanical Properties to IoTM Substitution

Mechanical characteristics such as elastic constants (relating stress to strain), viscoelastic parameters (relating stress, strain, and time), plastic parameters (permanent deformation), strength, ultimate properties (conditions for fracture or failure), and solubility can be tested to determine the role of IoTM on each biomechanical feature to determine the impact of IoTM on each property and possible correlations with the structural changes.

### Mechanical Characteristics of IoTM-Doped HAp

### Hardness and Plastic Parameters.

Dentin is a heterogeneous material; thus, nanoindentation can be used to determine microhardness, elastic constants, and viscosity of dentin. Additionally, nanoindentation provides for characterization of isolated components from microstructures or anatomical features of interest, enabling accurate indentations in a specific selected area.

Samples can be subjected to nanoindentation using a nanoindentation system. The nanoindentations can be performed three times on each sample and observed with scanning electron microscopy (SEM). Three indentations are a good measure for a standard deviation (SD) less than 15%, but can be increased to five for a larger observed SD. An average microhardness value (MHV) can be considered representative for each depth. Specimens after indentation can be prepared for SEM at 10 kV and 1 nA of probe current. SEM images can be standardized at 480×666 pixels. The cracks resulting from a nanoindentation process on the surface can be calculated with Image J. The specimens can be reused for solubility testing. This test can also be performed using a creep loading mode instead of a conventional loading-unloading procedure, and the displacement affected by creep can be corrected to derive the corrected contact stiffness (Sc), which will help derive elastic constants, the stress-strain curve, and Poisson's ratio.

### Microstrain

Microstrain in HAp crystals resulting from cationic substitution can be investigated using XRD to collect diffraction patterns from powdered samples. Small crystallites and microstrain result in broadening of HAp diffraction peaks. It is possible to modify the Scherrer equation to account for both crystallite size and microstrain broadening. A full width half maximum (FWHM) can be accurately determined through fitting techniques and data analysis. Microstrain is determined with the use of a Williamson-Hall plot, where β cos (θ) is plotted versus sin (θ) for all of observed HAp diffraction peaks. The Y-intercept is the crystallite size corrected for microstrain and the slope is the microstrain. Microstrain values between the substituted HAp and control HAp can be compared to assess the effects of cationic substitution.

### Dynamic Stress and Viscoelastic Analysis

Dentin is viscoelastic. Increased viscoelasticity elevates the ratio of minimum to maximum values of the stress wave and the phase shift between the stress pulse and load wave. This makes the material less vulnerable to an external load. In the present application, a finite element model can be developed by allocation of average geometry and mechanical properties to its components. The resultant static and dynamic stress values can be evaluated in pathologically important locations. Values from nanoindentation testing can be used to determine the viscoelasticity of dentin, where it has been showed that viscosity is determined by dividing hardness by a creep rate. A sensitivity analysis can study the effects of viscoelastic property and density of components on resultant dynamic stresses. Using a basic Kelvin model, loss and storage moduli can determine viscous and elastic parameters of a combined viscoelastic model, represented by dashpot and spring elements. A viscoelastic parameter is defined as the ratio of viscous (loss) to elastic (storage) moduli and can be calculated. The major variable is the viscoelastic parameter of dentin. (1) Static loading: Considering occlusal loads, a load of 135 N can be applied at the pit in the central buccolingual section of the model. The direction of load will be either parallel to the longitudinal axis of a tooth or 45° oblique from the axis; and (2) Dynamic loading: assuming that the load oscillates between 0 and a maximum of 135 N, the average period of loading is 0.8 s. The points of action of both static and dynamic loads are assumed to be identical. Static and dynamic loads can be introduced, and the resultant Von Mises stresses can be evaluated as a criterion of fracture based on principal stresses. For dynamic stress analysis, the amplitude and maximum value of stress waves can be obtained, and their dependency on viscoelastic properties can be analyzed. The phase differences between stress and load waves at different sites can characterize stress wave propagation.

### Effect of IoTM on the Solubility of Doped-HAp, to Ascertain if IoTM Solutions Used for Different Durations Degrade Dentin Material

Knowledge of the stoichiometries and thermodynamic solubility products of dentin minerals can be used to assess the driving forces for de- and re-mineralization due to IoTM by using pH cycling of teeth (to mimic oral conditions), followed by solubility testing. Fortified dentin specimens can be subjected to a pH cycling procedure with repeated sequences of de/remineralization. Dentin specimens can undergo 24-hour immersion in 2.5 ml of demineralizing solution (1.5 mM CaCl₂), 0.9 mM KH₂PO₄, 50 mM acetate buffer, pH 4.8). Samples can then be immersed in 2.5 ml of a remineralizing solution (1.5 mM CaCl₂), 0.9 mM KH₂PO₄, 20 mM HEPES [4-(2-hydroxyethyl)-1-piperazineethanesulfonic acid], pH 7.0) for 24 hours. The specimens can then be rinsed with deionized water for 20 seconds during each solution change. The pH cycling process can be performed at 37° C. for two weeks. Fortified dentin specimens can be completely immersed in 30 ml of 0.1 M butyric acid buffered with synthetic tissue fluid (pH 4.4) for 24 hours at 37° C. and 100% humidity. After incubation, the specimens can be removed from the acidic solutions, rinsed with distilled water to remove residual acid, and dried. The weight of the dentin slices can be measured by a digital microbalance before and after immersion into butyric acid, and the difference in weight will inform dentin solubility.

The nanoindentation test has the potential to introduce errors due to material inhomogeneity, which will be mitigated by selecting appropriate regions for testing and verifying results. For microstrain, instrumental resolution, sample preparation, and accurate peak fitting can be closely monitored to ensure reliable results The dynamic stress and viscoelastic analysis method may introduce uncertainties due to the assumptions of loading conditions and material properties, which can be addressed through sensitivity analyses.

### Relationship Between the Structural and Mechanical Effects of Trace Mineral Substitution in HAp to Demonstrate a Predictive Ability for Optimizing IoTM Substitution to Achieve Desired Properties

There is a complex and multivariable relationship between the type, size, and exposure of IoTM and the structural and mechanical changes as a result of cationic substitution. Two modeling efforts can be used to investigate these relationships and develop a capability to enable control of desirable properties based on IoTM inputs.

Regression can be applied to examine the relationship between structural and mechanical properties and machine learning to identify the multivariable patterns vs. IoTM input. Results may eliminate the need for additional expensive tests and provide a working model for future studies of the effect of IoTM on dentin. The data collected as described above can be used to develop these models.

### Regression Model to Find the Relationship Between IoTM-Doped HAp Structural and Mechanical Properties.

Regression algorithms used in machine learning that support multiple outputs directly include: (i) linear regression: models a linear relationship between inputs and outputs using a disturbance term or error variable to add noise; (ii) nearest neighbors: used in cases where the data labels are continuous rather than discrete variables. Basic nearest-neighbor regression uses weighting to compute a query point based on the labels of the nearest neighbors; (iii) decision trees: nonparametric supervised learning method that predicts the value of a target variable by learning simple decision rules inferred from the data features; and (iv) random forest: a meta estimator that fits a number of classifying decision trees on various sub-samples of the data set and uses averaging to improve the predictive accuracy and control over-fitting. These models can be tested with data sets and a k-fold cross-validation score can be used to obtain an unbiased estimate of model performance to select the model with the best performance.

Experimental Procedure: To Investigate the Structural and Mechanical Property Changes in Dentin Resulting from Trace Mineral Substitution in a Hydroxyapatite Lattice

A pH cycling experiment aimed to investigate the effects of AA2P salts of Sr, Ba, Zn, Mg, and Mn on the surface microhardness, compressive strength, diametral tensile strength (DTS), and solubility of root canal dentin was designed. Surface microhardness, compressive strength, diametral tensile strength (DTS), and solubility tests were conducted to evaluate the mechanical properties and demineralization resistance. Scanning electron microscopy (SEM) was performed to better understand the morphological changes in the dentin surface caused by the fortifying solutions. The null hypothesis was that no significant differences would be found between the saline and any of the tested fortifying solutions.

The mineralization quality of dentin plays a significant role in determining its mechanical properties, with microhardness being a key indicator. Microhardness is crucial in assessing initial signs of dentin caries and can be used to indirectly detect changes in composition and surface structure, reflecting mineral gain and loss in dental hard tissue. Mechanical response can also be evaluated using a diametral tensile strength (DTS) test, which provides reproducible results and assesses the tensile strength of friable materials. Compressive strength, determined through a similar methodology as DTS but with different specimen orientation, can be used for characterizing mechanical properties, particularly in terms of tooth restoration and treatment longevity.

One hundred and eighty six cylindrical dentin specimens from 93 teeth were fortified with optimal concentrations of AA2P salts of Mg (0.18 mM), Zn (5.3 μM), Mn (2.2×10⁻⁸ M), Sr (1.8 μM), and Ba (1.9 μM). Saline was used as the control group.

These dentin specimens underwent a 3-day cycling process simulating dentin caries formation through a repeated sequence of demineralization and remineralization. Surface microhardness at 100 and 500 μM depths (n=10/subgroup), scanning electron microscopy (n=3/subgroup), compressive strength (n=10/group), DTS (n=6/subgroup), and solubility (n=5/subgroup) tests were performed to analyze the dentin specimens.

Data were analyzed using Kolmogorov-Smirnov, one-way ANOVA, and Post Hoc Tukey tests (p<05).

### Material and Methods

### Ethical Considerations, Inclusion, and Exclusion Criteria

The Institutional Review Board approved a study protocol at Rutgers School of Dental Medicine. Ninety-three caries-free single-rooted premolars extracted for orthodontic purposes were included in this study. Teeth were collected from patients of both sexes between 20-40 years of age. Patients with a smoking history, a low-fat or vegetarian diet, or who were pregnant or lactating, were excluded from this study. Teeth were excluded from this study if they were preserved in antibacterial or fixative solutions, had cracks and defects (confirmed by using a stereomicroscope), or had previous root canal procedures or pathology. To disinfect the teeth, they were stored in 0.5% chloramine T at 4° C. for up to 15 days before use followed by storage in distilled water until tested.

### Specimen Size Analysis

G*Power 3.1.9.2 (Heinrich-Heine-Universität Düsseldorf, Düsseldorf, Germany) detected the total specimen size based on an effect size of 0.59 and an alpha-type error of 0.05 to achieve a power of 0.90. For evaluation of compressive strength, 10 specimens were used in each group (saline, AA2P salts of Mg, Zn, Mn, Sr, and Ba) (m=10/group). For the evaluation of microhardness, 20 specimens were used in each group (n=20/group). Each group was further subdivided into two subgroups according to the indentation depth (μm): 100 and 500 (n=10/subgroup). For SEM evaluation, the specimen size was determined by using the results of a pilot study. By comparing the means and standard deviation for the six groups for color intensity (control: 0.00±0.00; Mg: 9.9±2.12; Zn: 7.35±1.34; Ba: 5.55±1.34; Sr: 6.2±0.85; and Mn: 6.1±0.71), Applicants determined that the minimum number of samples in each group was 2. The specimen size was increased by 30% to improve the validity of the experiment. Considering an alpha-type error of 0.05 and a power of 80% (effect size=1.1), the final specimen size was estimated to be 3 per group for the unit of intensity test.

For the evaluation of solubility and DTS, the specimen size was also determined by using the results of a pilot study. By comparing the means and standard deviations for the six groups for weight loss (control: 32.51±0.036%; Mg: 12.61±5.67%; Zn: 12.55±3.82%; Ba: 18.96±3.44%; Sr: 18.16±4.21%; and Mn: 16.48±4.55%) and DTS (control: 40.60±3.37 Mpa; Mg: 57.60±15.21 Mpa; Zn: 53.98±9.94 Mpa; Ba: 61.96±4.47 Mpa; Sr: 59.99±1.91 Mpa; and Mn: 55.80±11.56 Mpa), Applicants determined that the minimum number of samples in each group was 4. The specimen size was increased by 20% to improve the validity of the invention. Considering an alpha-type error of 0.05 and a power of 90%, the final specimen size was estimated to be 5 per group for solubility and 6 per group for DTS tests.

### Specimen Preparation

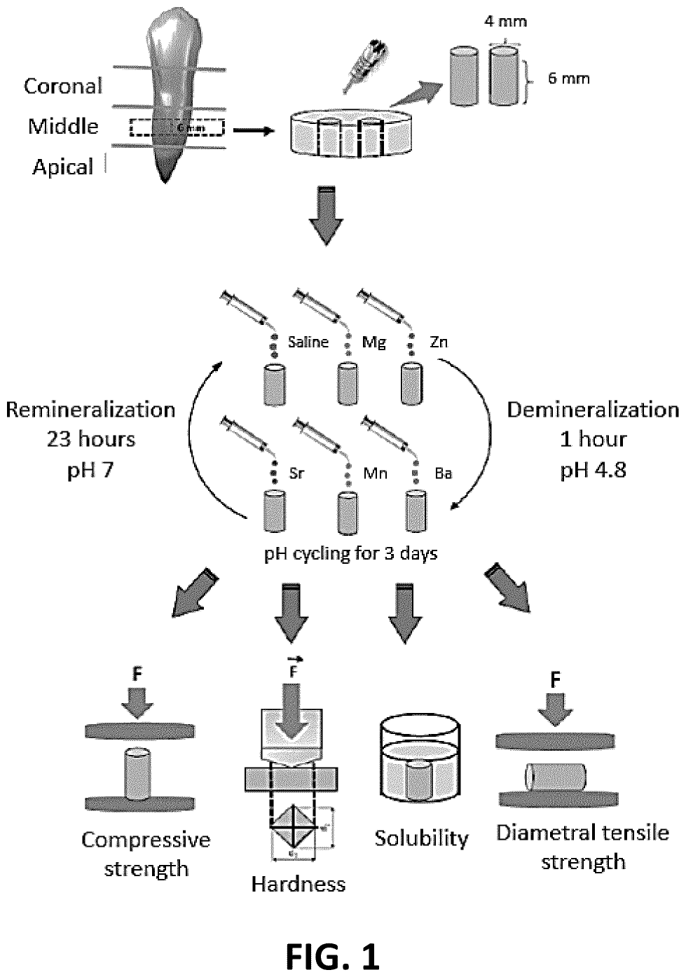

Routine access openings were prepared, and the working lengths were established 1 mm short of the apex. All of the canals were prepared up to file #35 with Protaper nickel-titanium rotary instruments and irrigated with 1% NaOCl while instrumenting the canals. Following root canal preparation, the canals were irrigated with 5 ml of 17% EDTA for 1 minute and flushed with 5 ml of saline solution. 93 dentin discs with 6 mm thickness were obtained from the mid-root region using a low-speed Isomet diamond saw under water-cooling. To obtain a standardized flat surface, the dentin specimens were wet-ground with a 320-grit and polished with a 600-grit under continuous water irrigation. Two cylindrical specimens (4×6 mm) were then instrumented from each dentin disc using a 6 mm trephine bur. A flow chart of the experiments design is shown in FIG. 1.

### Cationic Exchange

AA2P salts of the chosen metals (optimal concentration based on their ability to exchange 20% of the initial amount in dentin: Mg (0.18 mM), Zn (5.3 μM), Mn (2.2×10-8 M), Sr (1.8 μM), and Ba (1.9 μM)) were prepared by the following method: 3.22 g of AA2P trisodium salt was dissolved in 30 mL of water. The solution was then passed through a column packed with 100 mL of a strongly acidic cationic exchange resin (a gel-type polystyrene resin having a sulfonic acid group as an ion-exchange group; Amberlite IR-120B, H+ type), and the column was eluted with water to collect 150 mL of an effluent. The effluent was passed through a column packed with 120 mL of a weakly basic anionic exchange resin (a macroporous-type acrylic resin having a tertiary amine group as the ion-exchange group; Amberlite IRA-35) to obtain L-ascorbic acid only.

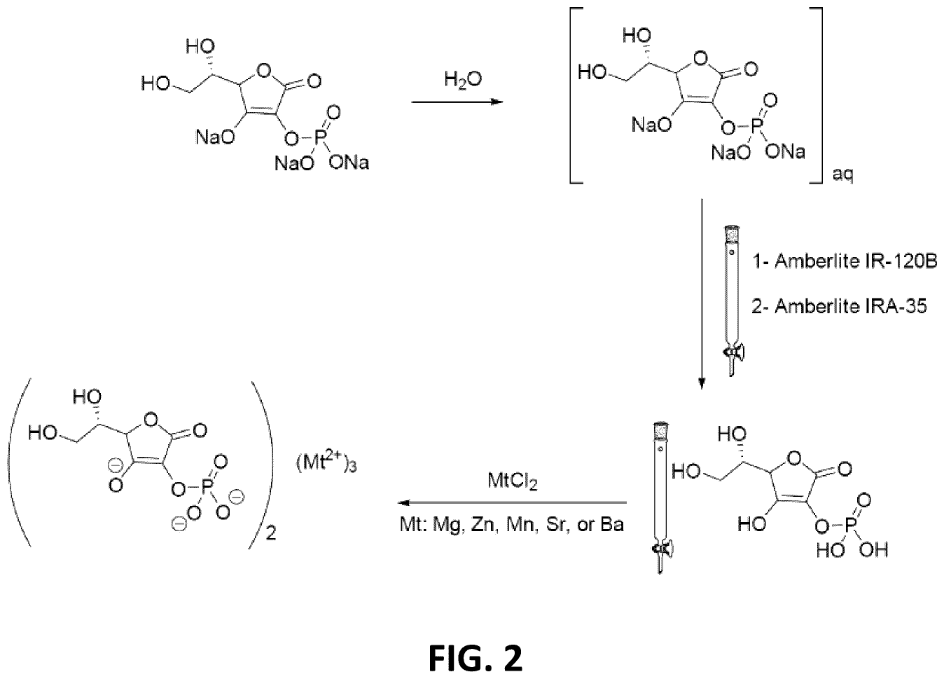

The column was further developed with 150 mL of one of five metal chloride solutions (0.1 mol/L AA2P salts of Mg, Zn, Mn, Sr, and Ba) to obtain a fraction containing new cationic L-ascorbic acid 2 phosphates with Mg, Zn, Mn, Sr, or Ba (FIG. 2). The prepared dentin specimens were randomly divided into six groups according to the solution used. The canal of each section was covered with an adhesive wax at the lower surface of the dentin specimen. Dentin specimens were then irrigated with one of five intervening solutions (AA2P salts of Mg (0.18 mM), Zn (5.3 μM), Mn (2.2×10-8 M), Sr (1.8 μM), and Ba (1.9 μM)) for five minutes, with the saline solution used as the control group. The root canals were then flushed with 10 mL of distilled water immediately after each treatment to remove excess solution.

### pH Cycling

Fortified dentin specimens were subjected to a pH cycling procedure with repeated sequences of de/remineralization. Artificial caries were formed in the dentin specimens by a 1-hour immersion in 2.5 mL of demineralizing solution (1.5 mM CaCl₂), 0.9 mM KH₂PO₄, 50 mM acetate buffer, pH 4.8). All lesions were then immersed in 2.5 mL of remineralizing solution (1.5 mM CaCl₂, 0.9 mM KH₂PO₄, 20 mM HEPES [4-(2-hydroxyethyl)-1-piperazineethanesulfonic acid], pH 7.0) for 23 hours. During each solution change, the specimens were rinsed with deionized water for 10 seconds. The pH cycling process was performed at 37 degrees Celsius for three days.

### Microhardness Measurements

A Micro Met 5100 durometer microhardness tester was used to measure the hardness of the fortified dentin specimens after exposure to a caries inducing environment. Each dentin specimen was further divided into two sections. Three separate indentations were made in each section at depths of 100 μm and 500 μm from the pulp-dentin interface using a 300-g load with a dwell time of 20 seconds for each measurement. The indentations were then evaluated under an optical microscope, and the average length of their two diagonal lengths was used to determine the hardness. The average microhardness value of the indentations' results was considered representative of each depth in each specimen.

### Scanning Electron Microscopy (SEM)

Three specimens from each group were examined by SEM-EDS mode to observe the precipitation of metallic components on the dentin surface. All specimens were sectioned at a depth of 500 μm (exposure surface to fortifying solutions) using a diamond-coated disk under water cooling. The specimens' surfaces were then polished and sputter-coated with 10 nm gold and observed under an SEM. An EDS color dot map analysis was performed for each specimen at 1000× to evaluate the distribution of trace elements. The intensity of Mg, Zn, Mn, Sr, and Ba was measured using ImageJ software. The color maps were converted to binary, and their mean color intensity (0 to 255 scale) was recorded as a unit of intensity (UI).

### Diametral Tensile Strength (DTS)

The cylindrical specimens were positioned on a customized loading fixture to ensure that the 1.00 mm diameter loading pin aligned with the center axis of the dentin cylinder. Additionally, flat platens were used at both ends of the specimens to further ensure alignment with the testing surface. The specimens were then loaded horizontally into a universal testing machine, receiving a uniaxial load perpendicular to their long axes and parallel to the dentinal tubule orientation. A 1.00 mm diameter stainless steel plunger was used to apply downward pressure on the dentin surface at 1.0 mm/min crosshead speed. The maximal load applied to the plug at the time of dislodgement was recorded in Newtons, and the DTS was calculated using the formula

$${σ}_{d} = \frac{2 F}{π Dh \text{?}} ,$$

$$\text{?} \text{indicates text missing or illegible when filed}$$

where F is the force in Newtons, D is the diameter of the cylinder, and h is the height in mm.

### Compressive Strength

The procedure for compressive strength testing was the same as for DTS testing except for the orientation of the specimens in the universal testing machine. The loads were applied uniaxially, parallel to the long axes of the specimens, and perpendicular to the dentinal tubule orientation until the specimens fractured. To calculate the compressive strength (MPa), the failure load (N) was divided by the area of the specimen in contact with the load

$$( {σ}_{c} = \frac{F}{π {r}^{2}} ) .$$

### Solubility Evaluations

Fortified dentin specimens were completely immersed in 30 ml of 0.1 M butyric acid buffered with synthetic tissue fluid (pH 4.4) for 24 hours at 37 degrees C. and 100% relative humidity. After incubation, the specimens were removed from the acidic solutions, rinsed with distilled water to remove any residual acid, and dried. The weight of the dentin slices was measured by a digital microbalance before and after immersion into butyric acid, and the difference in weight provided information on dentin solubility.

### Statistical Analysis

Descriptive statistics were calculated for all outcomes. Normal distribution of the data was confirmed by the Kolmogorov-Smirnov test. One-way ANOVA was used to compare compressive strength, solubility, unit of intensity, and DTS among the six groups. A general linear model was performed to determine if the groups and depth affect the microhardness. Tukey HSD was used for the post hoc tests. IBM SPSS Statistics version 28.0 software was used for all data analysis. The significance level (2-sided) was set to 0.05 for all tests.

### Results

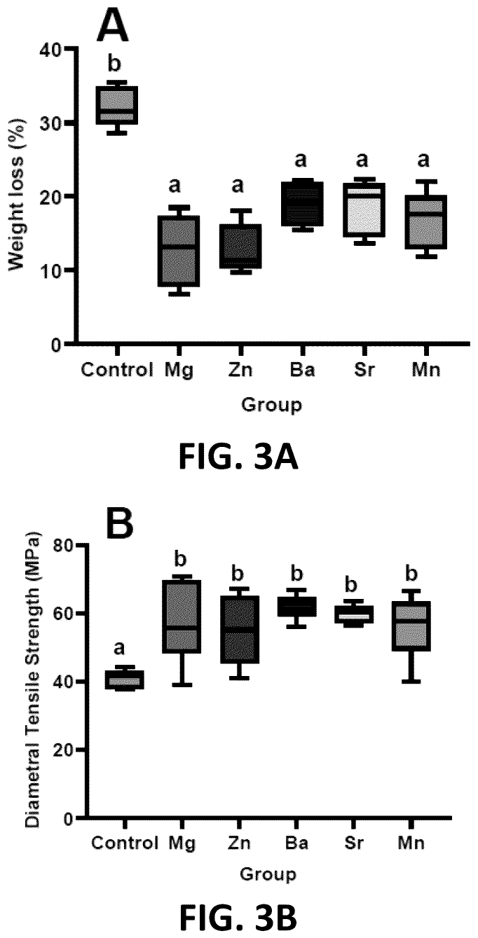

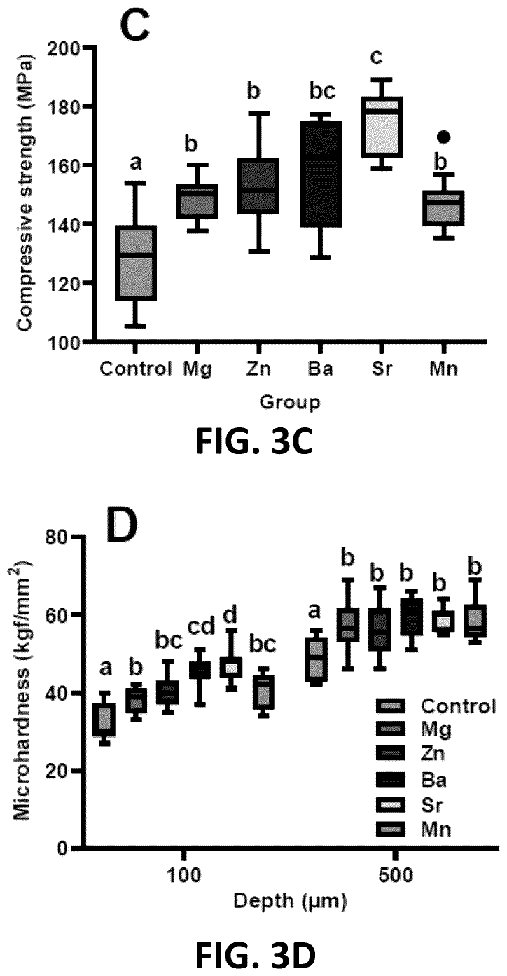

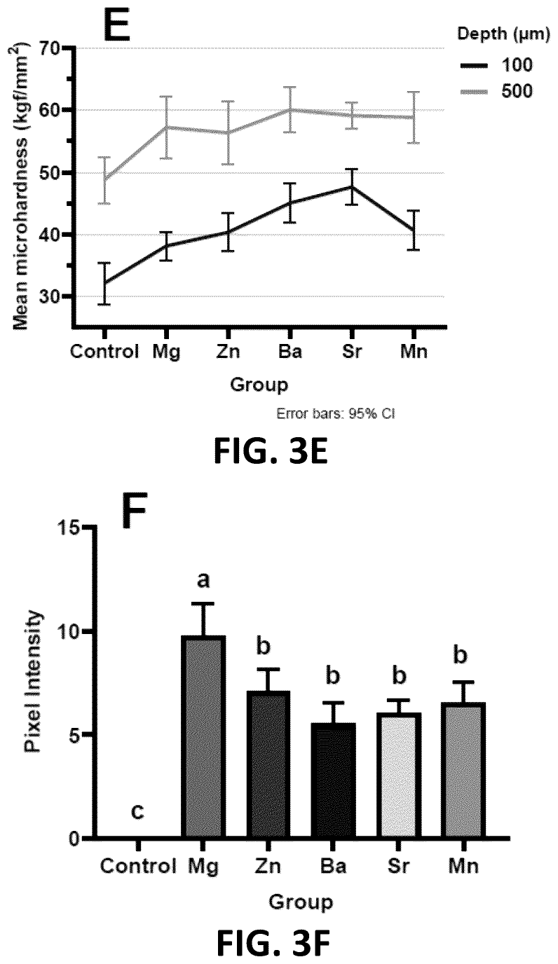

Applicant found a significant difference in solubility among the groups (p<0.001). The control group had significantly higher weight loss than all other groups (FIG. 3A). There was no significant difference between the non-control groups (Table 1).

TABLE 1
Descriptive Statistics Of DTS (Mpa), Solubility (%), Compressive Strength
(Mpa), and Unit of Intensity (Ui) and Comparison results between groups

| | Mechanical Properties | | | |
|---|---|---|---|---|
| | Diametral | | Compressive | |
| Groups | tensile strength | Solubility | strength | Unit of Intensity |
| Control | 40.99 ± 2.68 ^{a} | 32.18 ± 2.75 ^{b} | 127.21 ± 15.46 ^{a} | 0.00 ± 0.00 ^{c} |
| Mg | 56.93 ± 11.93 ^{b} | 12.73 ± 4.92 ^{a} | 148.97 ± 6.89 ^{b} | 9.76 ± 1.52 ^{a} |
| Zn | 54.97 ± 10.12 ^{b} | 12.88 ± 3.39 ^{a} | 152.40 ± 15.07 ^{b} | 7.1 ± 1.04 ^{b} |
| Ba | 61.56 ± 3.55 ^{b} | 19.03 ± 2.99 ^{a} | 157.96 ± 18.22 ^{bc} | 5.57 ± 0.95 ^{b} |
| Sr | 60.01 ± 2.70 ^{b} | 18.53 ± 3.74 ^{a} | 174.01 ± 11.09 ^{c} | 6.03 ± 0.67 ^{b} |
| Mn | 56.07 ± 9.39 ^{b} | 16.69 ± 3.97 ^{a} | 148.03 ± 9.82 ^{b} | 6.57 ± 0.95 ^{b} |

Note:
Different superscript letters indicate statistically significant differences between groups (p < 0.001, ANOVA and Tukey's HSD)

There was a significant difference in DTS among the groups (p=0.001). The control group had significantly lower DTS than all other groups (FIG. 3B). There was no significant difference between the non-control groups (Table 1). There was a significant difference in compressive strength among the groups (p<0.001). The control group had the lowest compressive strength and the Sr group had the highest compressive strength among all the groups (FIG. 3C). There was no significant difference among the Mg, Zn, Ba, and Mn groups, and between the Ba and Sr groups (Table 1). In Table 2, the general linear model showed that the groups (p<0.001) and depth (p<0.001) significantly affected the microhardness.

TABLE 2
Descriptive Statistics Of Microhardness
(kgf/mm²) for the fortifying solutions and depth,
and comparison results using a general linear model

| | | | | Std. de- | | Post hoc ** |
|---|---|---|---|---|---|---|
| | | N | Mean | viation | p-value | (Tukey HSD) |
| Groups | Control | 20 | 40.40 | 9.75 | <0.001* | a |
| | Mg | 20 | 47.65 | 11.11 | | b |
| | Zn | 20 | 48.30 | 9.96 | | bc |
| | Ba | 20 | 52.50 | 8.99 | | cd |
| | Sr | 20 | 53.35 | 6.87 | | bcd |
| | Mn | 20 | 49.70 | 10.59 | | |
| Depth | 100 | 60 | 40.62 | 6.41 | <0.001* | |
| | 500 | 60 | 56.68 | 6.61 | | |
| Groups*Depth | | | | | 0.211 | See |
| | | | | | | FIG. 3A- |
| | | | | | | FIG. 3F |

*The mean difference is significant at the 0.05 level.
** Same letter means that the mean difference is not significant between the groups.

The control group had the lowest microhardness and the Sr group had the highest microhardness among all the groups. However, there was no significant difference between the Sr, Ba, and Mn groups. The microhardness for 500 μm depth was significantly higher than the one for 100 μm depth. The general linear model also showed that the interaction of groups and depth variables was not significant (p=0.211, Table 2), which means that the difference in microhardness between the two depths was not significantly different across the groups (FIG. 3E). For the 100 μm depth, the microhardness was compared among the groups (Table 3).

TABLE 3
Descriptive Statistics Of Microhardness
(kgf/mm²) for the groups by depth, and comparison
results for each depth separately using one-way ANOVA

| | | | | Std. | | Post hoc ** |
|---|---|---|---|---|---|---|
| | | N | Mean | deviation | p-value | (Tukey HSD) |
| 100 μM | Control | 10 | 32.10 | 4.63 | <0.001* | a |
| depth | Mg | 10 | 38.10 | 3.18 | | b |
| | Zn | 10 | 40.30 | 4.24 | | bc |
| | Ba | 10 | 45.00 | 4.42 | | cd |
| | Sr | 10 | 47.60 | 4.12 | | d |
| | Mn | 10 | 40.60 | 4.48 | | bc |
| 500 μM | Control | 10 | 48.70 | 5.10 | <0.001* | a |
| depth | Mg | 10 | 57.20 | 6.93 | | b |
| | Zn | 10 | 56.30 | 7.01 | | b |
| | Ba | 10 | 60.00 | 5.10 | | b |
| | Sr | 10 | 59.10 | 3.04 | | b |
| | Mn | 10 | 58.80 | 5.73 | | b |

*The mean difference is significant at the 0.05 level.
** Same letter means that the mean difference is not significant between the groups.

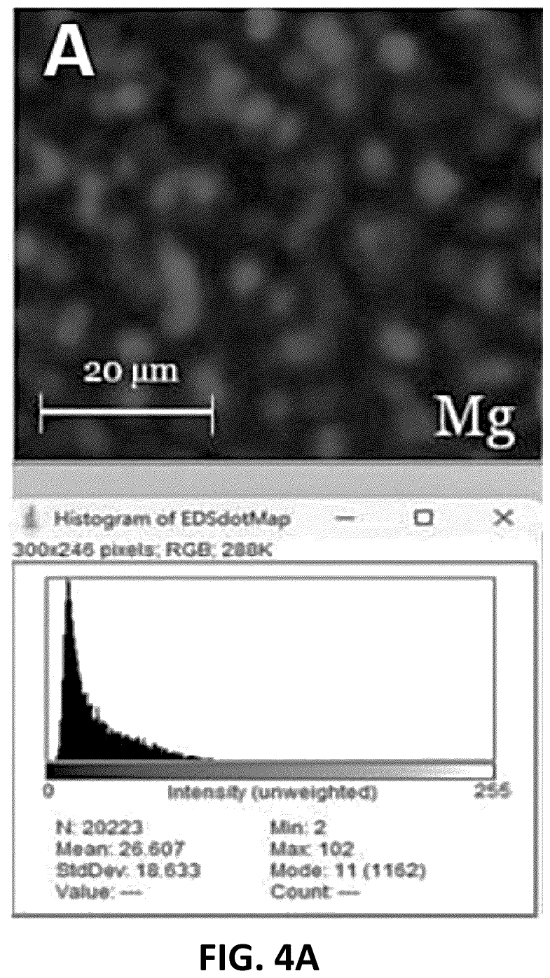

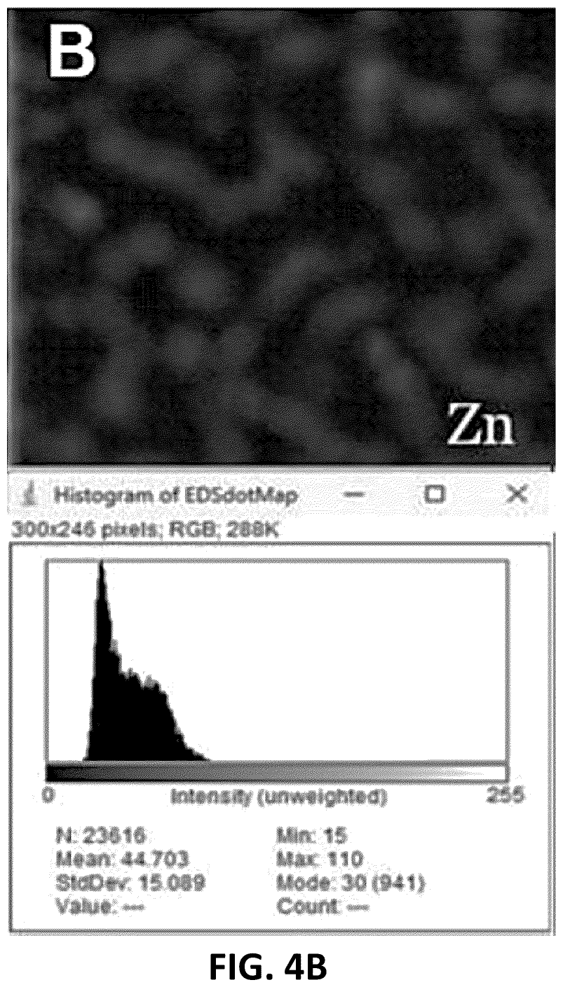

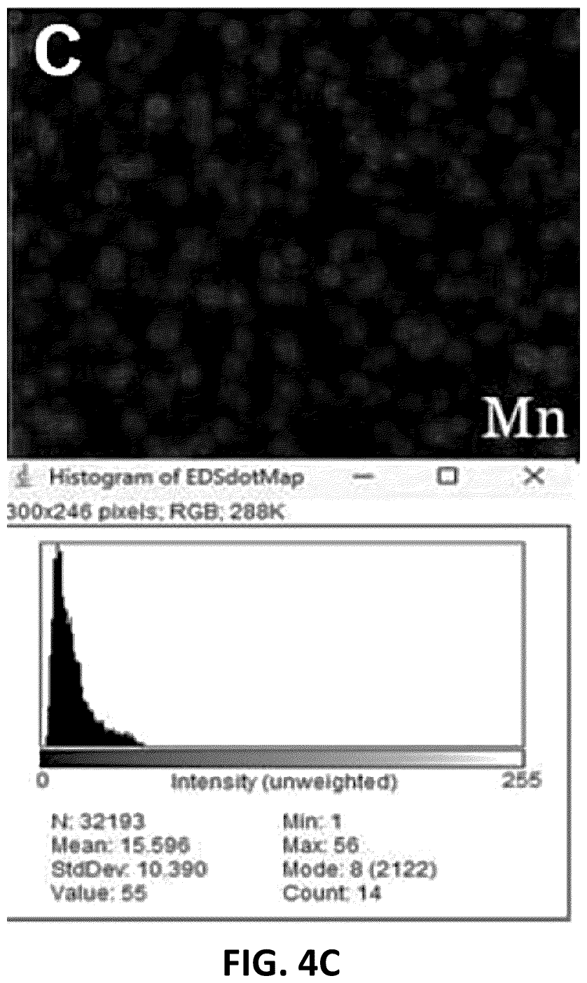

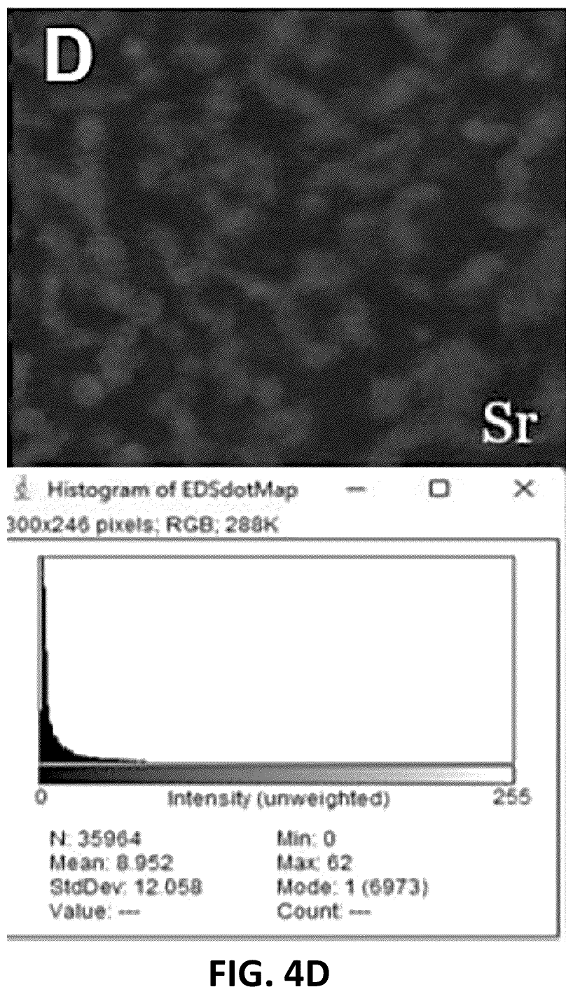

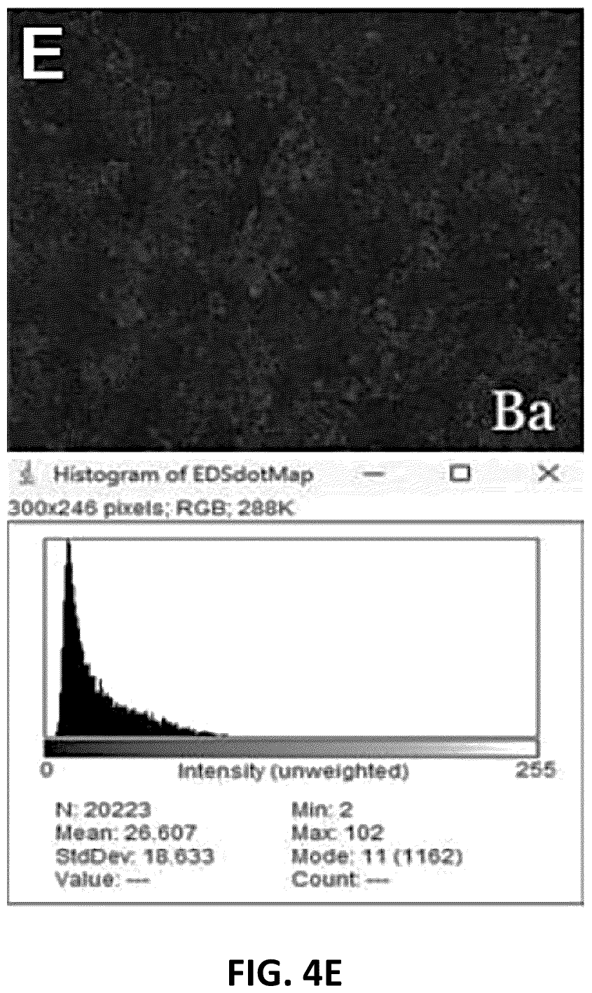

The result was similar to the combined data of 100 and 500 μm depth. The groups significantly affected the microhardness (p<0.001). The control group had the lowest microhardness and the Sr group had the highest microhardness among all the groups. There was no significant difference between the Sr and Ba groups. For the 500 μm depth, the group variable was also significant (p<0.001). The control group had significantly lower microhardness than all other groups. There was no significant difference between the non-control groups (FIG. 3D). There was a significant difference in the unit of intensity among the groups (p<0.001). The control group had the lowest intensity and the Mg group had the highest intensity among all the groups (FIG. 3F and FIG. 4A-FIG. 4E). There was no significant difference among the Zn, Mn, Sr, and Ba groups (Table 1).

In the present disclosure, Applicant used a pH cycling model to evaluate the effect of different fortifying solutions on dentin caries by assessing changes in surface microhardness, DTS, compressive strength, and solubility, which may reflect the mechanical properties and degree of dentin demineralization. According to the results of the experiments carried out, the null hypothesis was rejected. The optimal duration for irrigating the specimens with each fortifying solution was determined based on Applicant's previous study measuring the physical properties of dentin (e.g., surface microhardness) after treatment with Mg, Sr, and Zn solutions. Irrigation durations of 1, 2, 5, and 10 minutes were selected to align with the average time endodontic irrigants are used, as Applicant aimed to develop fortified irrigants for teeth in the future.

The results demonstrated a time-dependent effect of these elements on dentinal properties, with a significant effect observed after 5-10 minutes. However, considering practical limitations in irrigant use, 5 minutes was chosen as the final irrigation time for the present disclosure.

Dentin consists of many hydroxyapatite crystals with various cations and anions incorporated in its lattice, forming apatites with improved biomechanical properties. To ensure that the experiments were not confounded by the substitution of anionic counterions, the use of salts that have the same anionic counterion in the hydroxyapatite structure was considered, such as hydroxide salts or phosphate ions. However, phosphates are insoluble and unsuitable for fortifying solutions/irrigants, while hydroxides are excessively basic, posing challenges in neutralizing the solutions and potentially leading to the formation of unwanted by-products. Another option was to utilize salts whose anionic counterions are very large and cannot be replaced in the hydroxyapatite structure. Accordingly, AA2P was selected due to its advantages over phosphates and hydroxides. In particular, as the size of the anionic counterion increases, displacement occurs solely through the exchange of the cation. As a result, the concentration gradient created by this displacement will eventually reach equilibrium, ultimately enhancing the penetration rate.

Dentin specimens were treated with optimal concentrations of AA2P salts of Mg (0.18 mM), Zn (5.3 μM), Mn (2.2×10-8 M), Sr (1.8 μM), and Ba (1.9 μM)) for five minutes, with the saline solution used for the control group. The molarities of the AA2P salts were determined by taking into account the charge of the metal cation (+2) and the AA2P anion (−3), resulting in an adequate molar solution for each metal. The cation and anion displacement ensured charge neutralization throughout the process. The concentrations were selected to be 20% of the amount found in dentin to ensure uptake in dentin specimens while staying within the limits of the concentration safely allowable in the body. Increasing the concentration by 10-20% can improve bone tissue efficiency, but there are limits to the number of exchangeable surface elements, with a maximum of 30% for some elements. Thus, 20% was considered an appropriate concentration for this disclosure.

The present pH cycling model was a reliable method to simulate the acid-mediated demineralization process of root dentin and enamel surfaces. Dentin is more susceptible to caries than enamel due to factors such as a higher critical pH, faster demineralization, slower remineralization, and greater permeability to acids. To account for the structural and compositional variations between enamel and dentin, the number of pH cycles was reduced from 10 to 3. Moreover, the immersion time of the specimens in the demineralizing solution was shortened from 6 hours to 1 hour, while the remineralizing solution exposure time was increased from 18 to 23 hours.

The degree of demineralization in artificial root caries lesions is typically evaluated using polarized light microscopy and microradiography. However, polarized light microscopy provides only qualitative information, and microradiography is a destructive and time-consuming technique. In this disclosure, surface microhardness testing was selected because it reflects the initial signs of dentin caries, and can indirectly reveal mineral gain or loss in dental hard tissues by detecting changes in their composition and surface structure.

DTS was used to evaluate the mechanical response of materials under diametrically applied stress due to its relative simplicity and reproducible results. Additionally, it is the most widely used test for assessing the tensile strength of friable materials because it avoids the difficulties inherent to the flexural tensile strength test. Compressive strength is also useful for characterizing mechanical properties and follows a similar methodology to DTS, except for the orientation of the specimens in the universal testing machine.

In the present disclosure, dentinal mechanical properties, including surface microhardness, DTS, and compressive strength, were significantly lower in the control group compared to the Mg, Zn, Sr, Ba, and Mn groups. Lowered pH levels, below the critical range of 6.2-6.4, can result in increased mineral content loss from dentin, which leads to reduced tensile strength and hardness. A lower tensile strength in carious dentin compared to sound dentin, and a positive correlation between the tensile strength and hardness of carious dentin could be attributed to the weakened demineralized dentin matrix.

The fortifying solutions used, which contained Mg, Zn, Sr, Ba, or Mn, showed promising results in protecting against artificial caries lesions and improving the mechanical properties of carious dentin. The compressive strength and hardness varied significantly among the groups (p<0.001), with the Sr group having the highest compressive strength and hardness and the control group having the lowest. This improvement in strength may be attributed to the conversion of hydroxyapatite into Sr apatite, which enhances acid resistance, and the remineralization of dentin tissue through newly formed apatite. The Sr, Mg, Zn, Mn, and Ba groups had significantly higher DTS than the control group (p=0.001), but no significant differences were found between them. The protective effect of fortifying solutions on carious dentin was attributed to the higher mechanical properties, which could be related to the stabilizing effect of Mg on amorphous calcium phosphate and the higher compressive strength, toughness, hardness, and density of Mn-doped hydroxyapatite compared to pure hydroxyapatite.

SEM analysis further confirmed that the metallic components of the fortifying solutions penetrated through the dentin to strengthen the tissue, with Mg exhibiting the highest penetration at the 500 μm depth (FIG. 4A-FIG. 4E). Zn and Ba solutions also protected the dentin lesions. These solutions significantly improved the surface microhardness at depths of 100 μm and 500 μm (p<0.001), suggesting their ability to penetrate and strengthen the demineralized dentin against acid dissolution. Zn enhances the bioactivity and remineralization of dentin tissue by increasing Ca deposition and simulating protein phosphorylation and has shown promising potential to treat radicular dentin with oxipatite, a zinc oxide-modified hydroxyapatite. Ba promotes greater remineralization and fracture healing in bone tissue, which may extend to dentin tissue as observed in this disclosure, given the physiological and mechanical similarities between the two tissues. Solubility testing is a reliable and simple method for evaluating the chemical stability and susceptibility to demineralization of dental hard tissues, making it well-suited for investigating the effects of inorganic trace minerals on dentin solubility. The present disclosure used butyric acid for solubility testing to simulate the clinical conditions associated with dental caries formation, as it is a reported by-product of anaerobic bacterial metabolism. Applicant's results demonstrated that Mg, Sr, Zn, Ba, and Sr solutions provided 2-2.5 times greater resistance to dentin solubility than the saline solution. Mg increases the stability of dentin hydroxyapatite, while Zn promotes dentinal tubule occlusion through crystal precipitation that resists dental caries progression. Sr may assist in achieving a balance between dentin demineralization and remineralization, whereas Mn and Ba, known to be involved in osteoblast proliferation, differentiation, and metabolism in bones, may explain the lower solubility of carious dentin under low pH conditions in this invention. The limitations of this in vitro study include the absence of salivary and microbial influences that are typically present in natural caries lesions, as well as a relatively small specimen size. This study was also limited by the lack of a sound dentin control group, which could have provided information on whether the mineralizing treatments restored the baseline mechanical properties of dentin. Moreover, the results only featured some of the properties that contribute to the potential effectiveness of AA2P salts as caries prevention agents.

Future studies could explore the antibacterial activity and biocompatibility of AA2P salts, as well as use advanced characterization techniques such as atomic force microscopy and transverse microradiography to evaluate dentin surface roughness and mineral content, respectively. Future directions for this research could include identifying the optimal combination of trace minerals and exposure times to achieve the desired mechanical properties of dentin. Furthermore, it would be interesting to investigate the effects of AA2P salts on soft tissues, such as the pulp cells and other cells in the vicinity, to assess their cytocompatibility and biological impact on periapical tissues. All of the AA2P salts provided some protection against artificial caries lesions, suggesting that Mg, Zn, Sr, Ba, and Mn could have penetrated and strengthened the demineralized dentin against acid dissolution.

The control group had significantly lower microhardness at both depths (p<0.001), reduced DTS (p=0.001), decreased compressive strength (p<0.001), and higher weight loss (p<0.001) than all other groups. The Sr group had the highest compressive strength and microhardness among all the groups. The microhardness was significantly higher for the 500 μm depth than the 100 μm depth (p<0.001), but the difference in microhardness between depths across groups was not significant (p=0.211). All fortifying solutions provided some protection against artificial caries lesions. Therefore, these elements might have penetrated and reinforced the demineralized dentin against acid dissolution.

In some embodiments, as detailed above, the treatment of the present invention results in at least one of the following benefits: remineralization of dentin by enhancing the remineralization of dentin by doping it with divalent metal cations, which helps restore the mineral content and structural integrity of the dentin; improvement of surface microhardness by improving the surface microhardness of the dentin, making it more resistant to wear and acid dissolution compared to untreated dentin; increase in diametral tensile strength (DTS) by increasing the diametral tensile strength of the dentin, enhancing its ability to withstand tensile forces and reducing the risk of fractures; increase in compressive strength by increasing the compressive strength of the dentin, making it more robust and capable of withstanding compressive forces, which is important for the longevity and durability of dental restorations; enhanced penetration and reinforcement by the use of an ascorbic acid phosphate counterion and a silane coupling agent facilitates the effective penetration of the composition into the dentinal tubules, reinforcing the dentin structure and providing long-lasting protection against caries; biocompatibility and safety by employing orally acceptable salts and biosafe components, ensuring that the treatment is safe for use in the oral cavity and does not cause adverse reactions; versatility in application because the method can be applied in various forms, such as toothpaste, gel, dental cream, mouth-wash, mouth-rinse, chewing gum, lozenge, or food product, providing flexibility in its use for different dental care routines; and any combination thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An oral composition comprising:
(i) an effective amount of an orally acceptable salt comprising
an orally acceptable divalent metal cation and an anionic counterion,
wherein the anionic counterion comprises
an ascorbic acid phosphate counterion; and
(ii) a silane coupling agent.

2. The composition of claim 1, wherein the divalent metal cation is selected from strontium (Sr), barium (Ba), zinc (Zn), magnesium (Mg), manganese (Mn), or any combination thereof.

3. The composition of claim 2,
wherein the Mg is present in a concentration of 0.18 mM,
wherein the Zn is present in a concentration of 5.3 μM,
wherein the Mn is present in a concentration of 2.2×10⁻⁸ M,
wherein the Sr is present in a concentration of 1.8 μM, and
wherein the Ba is present in a concentration of 1.9 μM.

4. The composition of claim 1, wherein the divalent metal cation comprises from 10% to 15% by total weight of the composition.

5. The composition of claim 1, wherein the ascorbic acid phosphate counterion comprises L-ascorbic acid-2-phosphate (AA2P), L-ascorbic acid-3-phosphate (AA2P), or a combination thereof.

6. The composition of claim 4, wherein the ascorbic acid phosphate counterion is AA2P.

7. The composition of claim 5, wherein the AA2P comprises from 5% to 10% by total weight of the composition.

8. The composition of claim 1, wherein the silane coupling agent comprises 3-methacryloxypropyltrimethoxysilane (MPTS).

9. The composition of claim 7, wherein the MPTS comprises from 0.5% to 2% by total weight of the composition.

10. The composition of claim 1, further comprising a nano-zeolite.

11. The composition of claim 10, wherein the nano-zeolite comprises from 1% to 5% by total weight of the composition.

12. The composition of claim 1, which is formulated as a toothpaste, a gel, a dental cream, a mouth-wash, a mouth-rinse, a chewing gum, a lozenge, or a food product.

13. A method for treating a dentin, the method comprising:
contacting the dentin with an oral composition comprising
(i) an effective amount of an orally acceptable salt comprising
an orally acceptable divalent metal cation and an anionic counterion,
wherein the anionic counterion comprises
an ascorbic acid phosphate counterion; and
(ii) a silane coupling agent;
allowing the oral composition to remain in contact with the dentin for a predetermined duration; and
rinsing the dentin,
wherein the treatment results in at least one of the following: remineralization of the dentin, improvement of surface microhardness, increase in diametral tensile strength (DTS), increase in compressive strength of the dentin compared to untreated dentin or any combination thereof.

14. The method of claim 13, wherein the divalent metal cation is selected from strontium (Sr), barium (Ba), zinc (Zn), magnesium (Mg), manganese (Mn), or any combination thereof.

15. The method of claim 13, wherein the ascorbic acid phosphate counterion comprises L-ascorbic acid-2-phosphate (AA2P), L-ascorbic acid-3-phosphate (AA2P), or a combination thereof.

16. The method of claim 13, wherein the ascorbic acid phosphate counterion is AA2P.

17. The method of claim 13, wherein the silane coupling agent comprises 3-methacryloxypropyltrimethoxysilane (MPTS).

18. The method of claim 13, wherein the predetermined time is from 1 minute to 5 minutes.

19. The method of claim 13, wherein the treating comprises remineralization the dentin by doping with the divalent;
penetrating and reinforcing the dentin against acid dissolution compared to an untreated dentin;
improving a surface microhardness of the dentin compared to an untreated dentin;
increased a diametral tensile strength (DTS) of the dentin compared to an untreated dentin;
increasing a compressive strength of the dentin compared to an untreated dentin; or
any combination thereof.

20. The method of claim 19, wherein the surface microhardness is improved at a depth of from 100 μm to 500 μm.
